SEQUENCE LISTING (3 sequences)

SEQ ID NO: 1 - protein, 199 aa, Bos taurus
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 2 - protein, 169 aa, Bos taurus
AQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPKKNQDKTEIPTINTIASGEPTSTPTTEAVESTVATLEDSPEVIESPPEINTVQVTSTAV

SEQ ID NO: 3 - protein, 208 aa, Bos taurus
RELEELNVPGEIVESLSSSEESITRNKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

(19) United States
(12) Patent Application Publication
(10) Pub. No.: US 2010/0144044 A1
(43) Pub. Date: Jun. 10, 2010

(54) IDENTIFICATION OF PHOSPHORYLATION SITES IN POLYPEPTIDES BY EMPLOYMENT OF URANYL PHOTOCLEAVAGE

(75) Inventors: Niels Erik Mollegaard, Virum (DK); Peter Eigil Nielsen, Frederiksberg (DK)

Correspondence Address: KNOBBE MARTENS OLSON & BEAR LLP, 2040 MAIN STREET, FOURTEENTH FLOOR, IRVINE, CA, 92614, US

(73) Assignee: UNIVERSITY OF COPENHAGEN, Copenhagen N (DK)

(21) Appl. No.: 12/517,164

(22) PCT Filed: Dec. 7, 2007
(86) PCT No.: PCT/DK2007/050183

§ 371 Date: Dec. 3, 2009

(30) Foreign Application Priority Data
Dec. 7, 2006 (DK) PA 2006 01615

(51) Int. Cl. G01N 33/68; C07K 1/00; C07K 1/14

(52) U.S. Cl. 436/86; 530/345; 530/412

(57) ABSTRACT

The present invention relates to a method of cleaving a polypeptide at one or more phosphorylated residues. Said cleavage is induced by irradiation and is dependent on the presence of uranyl. The method is useful for analysis of phosphoproteoms and also for protein purification. The method also relates to a method of protein purification, wherein the phosphorylated protein is immobilized on a column said immobilization being dependent on uranyl.

### FIELD OF THE INVENTION

The present invention relates to analysis of phosphorylation sites in isolated polypeptides, polypeptides in cell extracts and analysis of the phosphoproteom of a cell. The invention further relates to affinity purification of phosphorylated polypeptides.

### BACKGROUND

Phosphorylation and dephosphorylation of proteins governed by the activities of thousands of kinases and phosphatases is crucial for controlling cellular function. Consequently, determination of the exact sites of phosphorylation has been an important step in experimental approaches involving control of any biological system.

Despite the importance of phosphorylation events in the cell, identification of the state and site of phosphorylation remains a challenge. In the past few years, the increased sensitivity of mass spectrometry has enabled the identifications of phosphorylation sites in the characterization of isolated proteins, cells or tissues.

However, of the estimated 100.000 phosphorylation sites in the proteome only a few thousand have been determined. Furthermore, identification of the phosphorylation site is not the only task in phosphorylation analysis since the extent of phosphorylation at a given site and in relation to other phosphorylation sites in the same protein is important for the biological function. Thus, quantitative and specific phosphoproteome analysis will be a major challenge in the years to come.

The state of the art procedure for analysing phosphorylation patterns in biological systems or in isolated protein is to digest a phosphoprotein or a phosphoproteome in a whole cell lysate with a protease (most often trypsin) to obtain smaller peptides for N-terminal sequencing and mass spectrometry analysis. Additionally, the procedure often includes phosphopeptide enrichment mainly by the employment of immobilized metal ion affinity chromatography (IMAC). This method is based on strong affinity of phosphopeptides for a metal ion (gallium, iron and others) chelated to a resin. By using this general approach many potential phosphorylation sites in proteins have been identified by sequencing and mass spectrometry followed by searches in protein databases to identify the sequences in the proteome.

All experimental strategies involving the proteome rely on the use of advanced and powerful mass analysis equipment. Several mass spectroscopy approaches have been used for detecting phosphorylated positions in the phosphoproteome by analysis of trypsin digested peptides. Among those is application of tandem MS in negative mode to scan for loss of the phosphate group, Maldi tof by inclusion of a phophatase step and LC-MS/MS by neutral loss of phosphoric acids and β-elimination.

Although tandem mass spectrometry has identified several phosphorylation sites, there are still limitations including signal suppression of phosphate containing peptides, lability of the phosphate group and complexity of achieving coverage of the full sequence of long peptides, peptides present in low amount and peptides phosphorylated at substoichiometric level.

In most cases, more than one mass analysis technique used in combination, have been a prerequisite for success in phosphoprotein analysis and it may vary from protein to protein.

Chemical modifications of phosphoaminoacids have been used for easier detection by mass spectrometry. For instance, the production of S-ethyl cysteine derivative of phosphoserine or the β-methyl S-ethyl cysteine derivative of phosphothreonine accomplishes identification by Edman sequencing.

An attractive goal has been to develop specific phosphoproteases which make the identification of phosphorylation sites and subsequent mass analysis and sequencing easier and much more informative. To obtain this goal, the S-ethyl cysteine derivative or the β-methyl S-ethyl cysteine have been converted into lysine analogues to generate targets for lysine specific proteases (Knight Z A, Schilling B, Row R H, Kenski D M, Gibson B W, Shokat K M. Nat Biotechnol. 2003 Sep.;21(9):1047-54).

Thus, instead of using a general protease digest, phosphospecific proteolysis generates specific cleavage at phosphoserine and phosphothreonine, which make the subsequent mass analysis more instructive. However, it is a very complicated procedure involving several complex chemical and enzymatic steps and the naturally occurring lysines will also interfere with the analysis.

In another strategy for quantitative phosphoproteome analysis, the phosphates were converted to phosphoramidate groups by coupling to a synthetic polyamine (dendrimer). The phosphopeptides were recovered by acid hydrolysis and analysed by mass spectrometry. This technique may have application in quantitative analysis of phosphorylation.

For analysis of global phosphorylation, the development of proteome chip technology has allowed the analysis of substrate specificity as for instance in analysis of specific protein kinases. This technique has been used for global analysis of phosphorylation in yeast. However, the technique does not solve the general problem namely easy detection of the extent and exact position of phosphorylation within the proteins.

Uranyl (UO₂²⁺) binds to the phosphates of DNA and RNA inducing cleavage of the backbone by oxidation of proximal deoxyriboses upon irradiation. Uranyl photocleavage has within recent years been used for studying protein-DNA interactions, drug-DNA interactions and the interactions of metal-ions with nucleic acids.

Uranyl cleavage has previously been reported for some non-phosphorylated proteins including bovine serum albumin (Duff M R Jr and Kumar C V, Angew Chem Int Ed Engl. 2005 Dec. 16; 45(1):137-9). The reported cleavage probably reflects coordination of the uranyl in the three-dimensional structure of the native protein.

### SUMMARY OF THE INVENTION

In a first aspect, the present invention relates to a method of cleaving a polypeptide at one or more phosphorylated residues. Said cleavage is induced by irradiation and is dependent on the presence of uranyl. A second aspect of the invention relates to a method of purifying a phosphorylated protein, wherein the phosphorylated protein is immobilized on a column, said immobilization being dependent on uranyl.

### BRIEF DESCRIPTION OF DRAWINGS

FIG. 1.

Uranyl photocleavage of phosphorylated (A) and dephosphorylated (B) α-casein. The uranyl/protein ratio is varied from approximately 0.025 to 2 from lane 2 to 8. The sample in lane 1 is irradiated without uranyl. The two bands appearing by cleavage of the phosphorylated form is indicated by arrows. It is noted that the total amount of protein disappear with increasing uranyl concentration, which is a result of precipitation. C. Sequence of α-casein

FIG. 2.

A: Uranyl photocleavage of phosphorylated k-casein. B: Uranyl photocleavage of phosphorylated β-casein. In A and B, the uranyl/ protein ratio is varied from approximately 0.025 to 8 from lane 2 to 8. The sample in lane 1 is irradiated without uranyl. The two bands appearing by cleavage of β-casein are indicated by arrows. It is noted that the total amount of protein disappear with increasing uranyl concentration, which is a result of precipitation. C: Amino acid sequence of κ-casein. D: Amino acid sequence of β-casein

FIG. 3.

Uranyl cleavage of β-casein in the presence of SDS, urea, and phosphate buffer Uranyl cleavage is done in 10 mM TrisHCl. 3A: Lane 1 is the full length protein, lane 2, cleavage in the absence of SDS. Lane 2-9 is uranyl cleavage in the presence of 0.01% to 1% SDS. 3B: Lane 1 is the full length protein, lane 2, cleavage in Tris HCL, no urea, lane 3, 4, 5 and 6 was 8, 6, 4 and 2 M urea, respectively. Lane 7, 8 and 9 Uranyl cleavage in the 50, 20 and 5 mM phosphate buffer

FIG. 4.

Purification of the phosphorylated α-casein with uranyl IMAC by employment of NTA columns from Qiagen, where the nickel ion is substituted by uranyl. Lane 1 is the run-though, lane 2 is the washing buffer (acetic acids), lane 3 and 4 is the eluate (phosphate buffer).

FIG. 5.

Purification of phosphorylated bovine α-casein added to an E. coli protein extract by immobilised uranyl affinity chromatography (IUAC).

The figure shows SDS-PAGE, where the proteins are visualized by silver staining.

The Lanes Show:

C: Protein extract, BSA and α-casein

R: Run-through

V1: Wash (water)

V2: Wash (water)

E1: Elution with 200 mM Na-citrate

E2; Elution with 0.1% SDS

E3: Elution with 1% SDS

### DETAILED DESCRIPTION OF THE INVENTION

The results of the examples section, demonstrate that uranyl binds specifically in a number of phosphorylated proteins and upon photocleavage, the protein is cleaved efficiently proximal to the phosphorylated position. This is the first example of specific cleavage at phosphorylation sites without any enzymatic or chemical modifications of the proteins and introduces an efficient and easy method for probing phosphorylation sites in isolated proteins and in the phosphoproteome.

The normal procedure in phosphorylation analysis is trypsin digestion before IMAC chromatography. Uranyl cleavage before IMAC is a better choice, because all the cleaved peptides has one single phosphoaminoacid, and therefore multiple phosphorylated peptides are not enriched, which is a general problem in IMAC

Thus, in a first aspect, the present invention provides a method of cleaving a polypeptide at one or more phosphorylated residues comprising the steps of:
a. Providing a sample comprising a phosphorylated polypeptide
b. Providing a sample comprising uranyl
c. Adding the sample comprising uranyl to the sample comprising the phosphorylated polypeptide to provide a uranyl-polypeptide sample
d. Irradiating the uranyl-polypeptide sample
e. Thereby photo cleaving the phosphorylated polypeptide at phosphorylated residues.

As used in herein, the term polypeptide refers to a string of amino acids and may thus be a short synthetic peptide, a protein, a fragment of a protein, the cleavage product of a protein etc.

As used herein, a phosphorylated polypeptide refers to a polypeptide comprising a phosphate group on one of its residues.

Typically, phosphorylated residues are serine residues. Also threonine and tyrosine residues may be phosphorylated. In special cases, also other residues may be phosphorylated.

In a preferred embodiment, the photocleavage is performed under denaturing conditions.

As demonstrated in the examples section, uranyl photo-cleaves efficiently at phosphorylated residues under denaturing condition. Thus, uranyl photo-cleavage at phosphorylated residues is not dependent on the tertiary structure of the protein.

Therefore, photocleavage under denaturing conditions has the advantage of reducing phosphorylation independent cleavage due to uranyl binding pockets in the three dimensional structure of a protein.

Denaturing conditions may also be used to reduce or eliminate enzymatic activities in the sample, such activities e.g. originating from phosphatases and proteases.

Denaturing conditions may be obtained by including SDS or urea in the uranyl-polypeptide sample.

In yet another embodiment of the invention, photocleavage is performed in the presence of a chelator of divalent cations. The presence of a chelator of divalent cations may be desirable e.g. to inhibit the activity of enzymes dependent on divalent cations. It may e.g. be desirable to inhibit the activity of phosphatases or proteases.

Preferably, the chelator is selected from the group consisting of: EDTA, EGTA, BAPTA, and citrate.

In a preferred embodiment, photocleavage is performed at a temperature selected from the group consisting of: between 0° C. and 4° C., between 4° C. and 20° C., between 20° C. and 37° C., between 30° C. and 42° C., between ° C. 42 and 52° C., between 52° C. and 70° C., between ° C. 70 and 94° C., and above 90° C.

The temperature may be adjusted such as to affect the three dimensional structure of the phosphorylated polypeptide, optionally in combination with the addition of denaturants to the sample. Also reaction efficiency may be affected by the temperature. As is well-known to the skilled man, increasing temperatures leads to increased reaction rates.

In a preferred embodiment of the invention, the light source used for irradiation emits light with maximum emission at a wavelength between 200-500 nm.

More preferably, the light source used for irradiation emits light with maximum emission at a wavelength between 300 nm and 450 nm and even more preferably at 420 nm.

In a preferred embodiment, irradiation is done in open tubes placed below a fluorescent light tube with maximum emission at 420 nm.

Photocleavage may cleave the phosphorylated polypeptide at the N-terminal site of the phosphorylated residue, at the C-terminal of the phosphorylated residue, or at both the N-terminal site of the phosphorylated residue and at the C-terminal of the phosphorylated residue.

In a preferred embodiment, phosphorylated polypeptide is cleaved at the N-terminal site of the phosphorylated residue.

Protein/Proteome Analysis

The first aspect of the invention will be a valuable tool not only for probing phosphorylation sites in isolated proteins, but also in the whole proteome of a cell or isolated parts of the proteom.

Thus, in a preferred embodiment of the first aspect, the sample comprising a phosphorylated polypeptide is a cell extract or is derived from a cell extract. When the sample is derived from a cell extract, it may e.g. have been subjected to chromatography (affinity, ion-exchange, gel-filtration etc.) or precipitations.

Engineered Proteins

A general problem in most tag-affinity purification of proteins is to cleave off the tag after affinity purification. We reasoned that this may be done by introducing a uranyl cleavage site (i.e. a phosphorylation site) between the affinity tag and the protein to be purified, such that the affinity tag may be cleaved off after affinity purification.

In a preferred embodiment, the uranyl cleavage site also serves as affinity tag, as the uranyl cleavage site has affinity for uranyl. Thus, a second affinity tag may be omitted.

Hence, in another embodiment of the invention, the sample comprises a genetically engineered polypeptide with an artificially introduced phosphorylation site uranyl cleavage site for directed cleavage.

In a preferred embodiment, the genetically engineered polypeptide makes up more than 50% w/w of the total polypeptides of the sample.

In another embodiment, the genetically engineered polypeptide makes up a percentage of the w/w of the total polypeptides of the sample selected from the group consisting of: more than 60%, more than 70%, more than 80%, more than 85%, more than 90%, more than 95% and more than 99%.

In still another preferred embodiment, the genetically engineered polypeptide further comprises an affinity tag. Preferably, the introduced phosphorylation site for directed cleavage is placed such as to enable removal of the affinity tag from the polypeptide.

Thus, in one embodiment one or more serines may be placed between e.g. a his-tag and the protein to be purified. It is to be understood that the affinity tag and uranyl cleavage site is fused to the protein to be purified using genetic engineering.

A wide range of affinity tags are known to the skilled man. Examples are the his-tag, the flag-tag and the GST-tag.

Chromatography

In a preferred embodiment of the invention, the sample comprising a phosphorylated polypeptide has been subjected to IMAC chromatography before photocleavage to enrich for phosphorylated polypeptides.

In another preferred embodiment of the invention, the photo cleaved sample is subjected to IMAC chromatography after photocleavage to enrich for phosphorylated polypeptides.

Analysis

In still another preferred embodiment of the invention, the method further comprises analysing the products of photocleavage to identify phosphorylation sites.

In a preferred embodiment, analysis involves mass spectrometry and/or N-terminal sequencing.

Preferably, when using N-terminal sequencing, phosphorylation sites are detected indirectly, because it is known that phosphorylated polypeptides are cleaved at phosphorylated sites.

When using mass spectrometry, phosphorylations may be detected directly by detection of extra mass.

Thus, in a preferred embodiment, the analysis is quantitative such as to determine the degree of phosphorylation at phosphorylated sites.

Quantitative analysis may e.g. be done using mass spectrometry, wherein the amount of a particular phosphorylated peptide may be compared to the amount of the same peptide with no phosphorylation.

On Column Cleavage

In a preferred embodiment of the invention, the uranyl-polypeptide sample is irradiated while immobilized on the IMAC column In this embodiment, products of the photocleavage that do not comprise a phosphate group will be released from the column, i.e. the released polypeptides can also be analysed such as to give information on phosphorylation sites in the polypeptide.

Thus, in a preferred embodiment, both the products released from the IMAC by photocleavage and the remaining phosphorylated polypeptides on the IMAC column are analysed.

In another preferred embodiment, only the remaining phosphorylated polypeptides on the IMAC column are analysed. Optionally, they may be eluted before analysis.

Method of Purifying a Phosphorylated Protein

A second aspect of the invention is a method of purifying a phosphorylated protein from a sample, wherein the phosphorylated protein is immobilized on a column via uranyl coordinated to one or more phosphorylated sites on the protein.

Instead of using a column, batch purification may be used. I.e. the same column material can be used in connection with a column and for batch purification. Thus, when referring to purification using a column or immobilization on a column, the terms also include batch purification and immobilization to the column material in batch. As the skilled man will recognize, column purification and batch purification can even be combined. E.g. a sample may be applied to the column material in batch, the column material may then be washed in batch and then applied to a column. After application to a column, further washing steps may be performed and finally an elution step may be performed.

In a preferred embodiment, the method of purifying a phosphorylated protein from a sample comprises the steps of:
a. Providing a column with affinity for uranyl.
b. Providing a sample comprising a phosphorylated polypeptide
c. Providing a sample comprising uranyl
d. Adding the samples of step b. and step c. to the column of step a. under conditions allowing immobilization of the phosphorylated peptide.
e. Removing non-binding polypeptides of the sample
f. Thereby purifying the phosphorylated protein.

In a preferred embodiment of the method, the sample comprising the phosphorylated polypeptide is first added to the sample comprising uranyl, where after the resulting sample is added to the column with affinity for uranyl.

In another preferred embodiment, the sample comprising uranyl is first added to the column, where after the sample comprising phosphorylated polypeptide is added to the column with affinity for uranyl.

Preferably, the column is any affinity column including immobilized metal ion affinity chromatography (IMAC) column that binds uranyl, i.e. the column is not saturated with cations.

Alternatively, the column is a phosphate column that binds uranyl. Thus, the phosphorylated polypeptide will bind to a uranyl ion that also binds to phosphate on the column.

In a preferred embodiment of the method of purifying a phosphorylated protein, the method further comprises a step of eluting the immobilized protein.

Elution may be done with e.g. a buffer comprising imidazole phosphate, high cation (e.g. uranyl), metal-chelators, acid and base.

Suspensions may be used instead of columns and elution done by batch elution, as also mentioned above.

In a preferred embodiment, the sample comprising a phosphorylated polypeptide is a cell extract or is derived from a cell extract.

In still another preferred embodiment, the sample comprising a phosphorylated polypeptide comprises a genetically engineered polypeptide with an artificially introduced phosphorylation site for directed cleavage.

In a preferred embodiment, the genetically engineered polypeptide makes up more than 50% w/w of the total polypeptides of the sample.

In another embodiment, the genetically engineered polypeptide makes up a percentage of the w/w of the total polypeptides of the sample selected from the group consisting of: more than 60%, more than 70%, more than 80%, more than 85%, more than 90%, more than 95% and more than 99%.

In still another preferred embodiment, the genetically engineered polypeptide further comprises an affinity tag. Preferably, the introduced phosphorylation site for directed cleavage is placed such as to enable removal of the affinity tag from the polypeptide.

A wide range of affinity tags are known to the skilled man. Examples are the his-tag, the flag-tag and the GST-tag.

### Examples

Material and Methods

N-terminal sequencing and mass spectrometry: Cleaved proteins are separated on SDS page and the identified fragments are N-terminally sequenced directly from the gel by blotting to a PVDF membrane on a Precise protein sequencing system. To identify the N-terminally sequenced fragments the peptides is eluted from the PVDF membrane and ESI-MS and MS-MS fragment analysis is performed. Alternatively the fragments are separated from cleaved proteins by HPLC coupled to ESI-MS for direct analysis

### Example 1

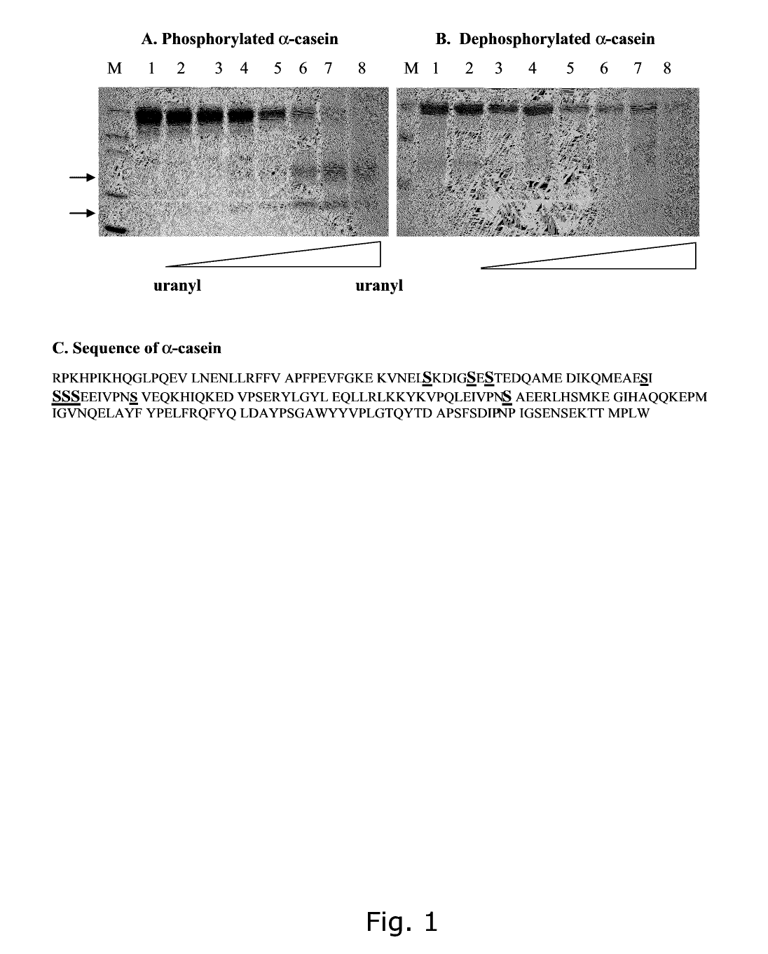

Initially we applied the uranyl cleavage analysis on a phosphorylated and a dephosphorylated form of α S1 casein. This protein of 25 kda contains several phosphorylated positions (FIG. 1C).

No cleavage was observed when the sample was irradiated in the absence of uranyl or incubated with uranyl without irradiation. However, a uranyl titration experiment in the presence of irradiation shows that the full-length protein is dose dependently and specifically cleaved into two bands as visualized on the SDS gel (FIG. 1A). The efficiency is remarkable since no full-length product remain at a uranyl/protein ratio of 2. It is noted that the total amount of protein decreases with increasing uranyl concentration, which may be an effect of protein precipitation or binding to the tube.

The absence of uranyl photocleavage of the non-phosphorylated form of α S1 casein clearly demonstrates that phosphorylation of the protein is a prerequisite for strong cleavage since no cleavage is observed in the dephosphorylated form of the protein, not even at the highest uranyl concentration.

The dramatic difference in uranyl cleavage between the non-phosphorylated α-casein compared to the phosphorylated protein clearly demonstrates that any background cleavage in this system is very low.

### Example 2

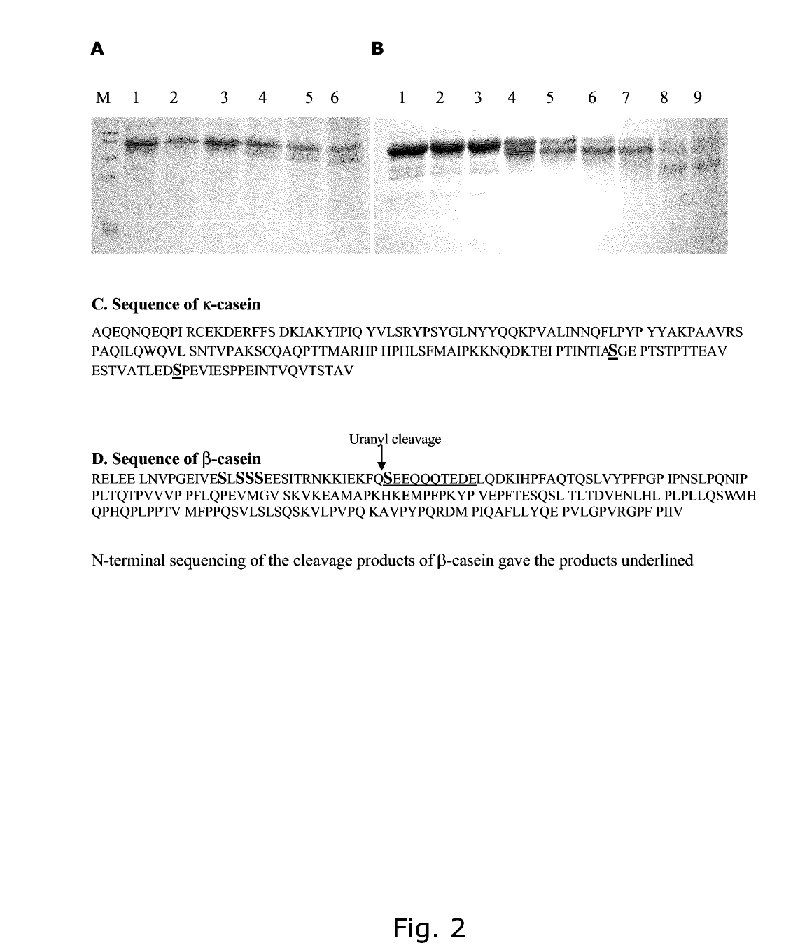

The α-casein used in example 1 contains eight phosphorylation sites, which complicate sequence and mass analysis because several products will have approximately equal masses. Therefore we chose to analyse phosphorylated proteins with fewer phosphorylation sites in order to identify the exact cleavage positions. Two other caseins were analysed. The κ-casein contains two phosphorylated amino acids close to the C-terminal end (FIG. 2C). Cleavage of the 23 kd full length product would cut approximately 2 and 4 kda off resulting in products of 19 and 21 kda. The uranyl cleavage of κ-casein shows highly selective cleavage revealing only one band appears on the gel (FIG. 2A). Most likely it is not possible to separate 19 and 21 kda products on this type of gel indicating that the band could represent the two products. It is noted that the cleavage efficiency is less significant than in α-casein, which may reflect the lower number of phosphorylation sites.

Likewise β-casein contains several phosphorylated serines (FIG. 2D). The native protein was cleaved with uranyl and the appearance of two bands clearly demonstrates efficient and specific uranyl cleavage. Interestingly the cleavage pattern is changing with increasing uranyl concentrations (FIG. 2B). It is noted that at lower concentrations of uranyl only one band appears, whereas two bands emerges at higher uranyl concentrations. Finally, at the highest concentrations the upper band disappears. An attractive explanation is that at lower uranyl concentrations only one or two positions are cleaved within each protein molecule. Thus, the upper band probably reflects cleavage at any of the four phosphoserines within the local sequence EIVESLSSSEESITR. Cleavage at the four sites results in four product of approximately 19 kda, which do not separate on an SDS gel. These four products are represented by the upper band. At higher concentrations of uranyl all five positions are probably cleaved. Thus only the lower band is observed representing cleavage in KFQSEEQ. This interpretation seems to be correct since N-terminal sequencing of the lower band interestingly show that uranyl indeed cleave exactly at the N-terminal side of the phosphoserine.

### Example 3

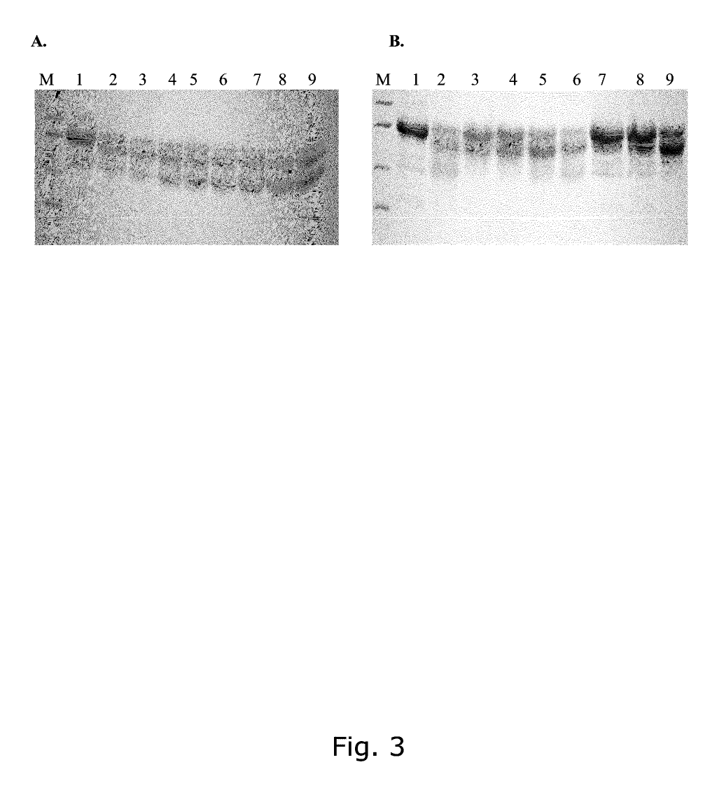

In order to analyse the effect of the protein folding on the uranyl reaction, β-casein was denatured prior to uranyl cleavage. Interestingly uranyl photo-treatment after denaturing by SDS and heating at 90° not only results in similar cleavage product, but also in increased cleavage (FIG. 3A).When cleavage is done in the absence of SDS in this particular experiment, the upper cleavage product accounts for almost all of the cleavage products (lane 2). However with increasing concentrations of SDS in the presence of heating, the lower product appears as well. It is noted that a similar experiment without heating gave the same result (not shown). Apparently, denaturing of the protein by SDS increases cleavage, which shows that the secondary/tertiary structure of the protein is not a prerequisite for specific and effective photocleavage. Noteworthy, it seems as if the negative SDS has no interaction with the uranyl ion indicating that the contact between the phosphoaminoacids and uranyl is remarkably strong.

To verify the effect of denaturation on uranyl cleavage an experiment with urea as the denaturing agent was performed. Three concentrations of urea were used and it is noted that at 8 M urea the cleavage is inhibited but still present (lane 3, FIG. 3B). The inhibitory effect of urea is most likely an effect on the uranyl reaction and not a result of denaturing according to the experiment with SDS and heating.

It would be expected that uranyl cleavage in a phosphate buffer would inhibit cleavage. It is noted that at 5 mM phosphate no inhibition is observed, at 20 mM the cleavage is almost fully inhibited. While at 50 mM the cleavage is absent. The effect of phosphate buffer on cleavage of α-casein was analysed as well (data not shown). It is noted that 20 mM phosphate decreases cleavage, whereas 50 mM totally inhibit cleavage. To further analyse the effect of potential inhibitors of the reaction an experiment with EDTA and citrate was performed. It is expected that uranyl creates strong complexes with citrate and EDTA. Therefore it is rather surprising that the two chelators have no effect on uranyl cleavage which still is efficient at 1 mM citrate and 1mM EDTA (data not shown). It is noted that the protein concentration is 20 μM and uranyl 25 μM.

Thus, uranyl apparently has very high affinity for the phosphate on the serines.

### Example 4

Immoblization of metal ions using chelating agents is widely used to purify phosphopeptides. The strong affinity for phosphates makes uranyl an obvious choice in IMAC, where the Ga³⁺ and Fe³⁺ linked to nitrilotriacetic acid (NTA) og imminodiacetic acid (IDA) sepharose are most efficient.

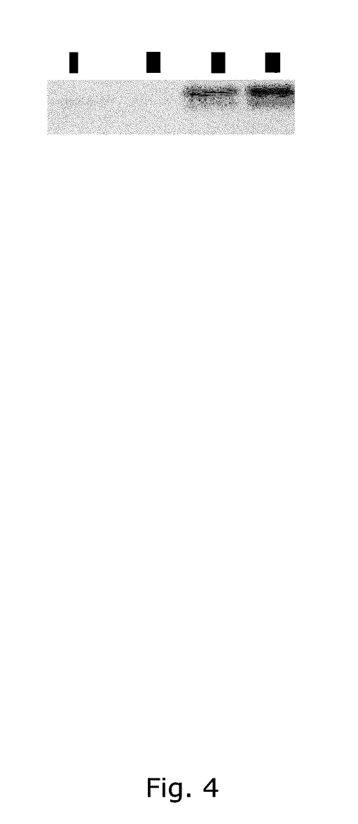

Therefore, we decided to exchange the nickel ion with uranyl in a Ni—NTA column. The result indicates that uranyl captures phosphorylated proteins (FIG. 4). Furthermore an experiment where the protein was cleaved before purification on IMAC with uranyl revealed that the fragments still containing the phosphate can be purified this way (data not shown). This opens up for easy IMAC and analysis of phosphorylation sites.

### Example 5

Purification of phosphorylated bovine α-casein added to an E. coli protein extract by immobilised uranyl affinity chromatography (IUAC).

Uranyl was coupled to O-phospho-L-serine-sepharose (Jena Bioscience), which was loaded on centrifugation filters. A protein extract from E. coli (no phosphorylation), Bovine serum albumin (BSA) and phosphorylated α-casein was added to the column.

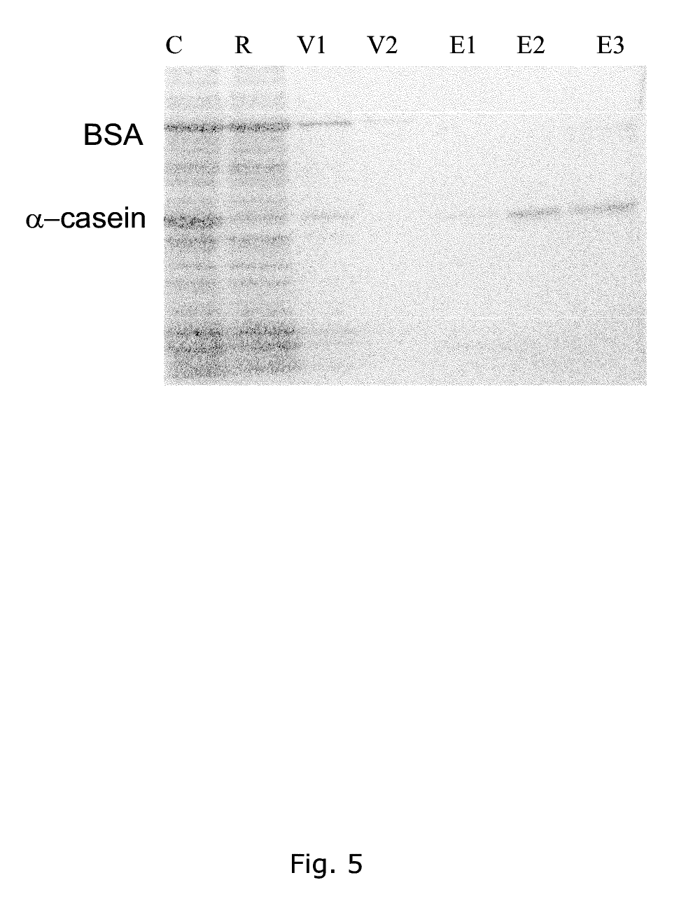

FIG. 5 shows SDS-page is shown, where the proteins are visualized by silver staining. The result shows that all proteins (non-phosphorylated) from the bacteria and the added BSA (non-phosphorylated) primarily are found in the run-through, whereas the α-casein is selectively found in the elution buffer. This shows that IUAC principally can be used for purification of phosphorylated proteins.

Conclusion

The examples show phosphospecific proteolysis without any chemical or enzymatic modifications of the phosphoaminoacids. It is supposed that phosphospecific proteolysis by uranyl photocleavage will be a valuable tool for fragmentations of proteins into peptides exactly at the position of the phosphate. According to the mechanism of cleavage in β-casein cleavage occurs N-terminal to the. Thus, N-terminal sequencing of the peptides together with mass spectrometry will identify the exact position of the phosphoaminoacids.

1. A method of cleaving a polypeptide at one or more phosphorylated residues comprising the steps of:
a. Providing a sample comprising a phosphorylated polypeptide;
b. Providing a sample comprising uranyl;
c. Adding the sample comprising uranyl to the sample comprising the phosphorylated polypeptide to provide a uranyl-polypeptide sample; and
d. Irradiating the uranyl-polypeptide sample, and thereby photo cleaving the phosphorylated polypeptide at phosphorylated residues.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The method of claim 1, wherein the photocleavage is performed under denaturing conditions.

32. The method of claim 31, wherein the denaturing conditions are obtained by including SDS or Urea in the uranyl-polypeptide sample.

33. The method of claim 1, wherein photocleavage is performed in the presence of a chelator of divalent cations.

34. The method of claim 33, wherein the chelator is selected from the group consisting of: EDTA, EGTA, BAPTA, and citrate.

35. The method of claim 1, wherein photocleavage is performed at a temperature range selected from the group consisting of: between 0° C. and 4° C., between 4° C. and 20° C., between 20° C. and 37° C., between 30° C. and 42° C., between ° C. 42 and 52° C., between 52° C. and 70° C., between ° C. 70 and 94° C., and above 90° C.

36. The method of claim 1, wherein the light source used for irradiation emits light with maximum emission at a wavelength between 200-500 nm, between 300 nm and 450 nm, or 420 nm.

37. The method of claim 1, wherein the polypeptide is cleaved at the N-terminal site of the phosphorylated residue.

38. The method of claim 1, wherein the sample comprising a phosphorylated polypeptide is a cell extract or is derived from a cell extract

39. The method of claim 1, wherein the sample comprise a genetically engineered polypeptide with an artificially introduced phosphorylation site for directed cleavage.

40. The method of claim 39, wherein the genetically engineered polypeptide makes up more than 50%, more than 60%, more than 70%, more than 80%, more than 85%, more than 90%, more than 95% or more than 99% w/w of the total polypeptides of the sample.

41. The method of claim 39, wherein the genetically engineered polypeptide further comprises an affinity tag.

42. The method of claim 1, wherein the sample comprising a phosphorylated polypeptide has been subjected to IMAC chromatography before photocleavage to enrich for phosphorylated polypeptides.

43. The method of claim 1, wherein the photo cleaved sample is subjected to IMAC chromatography after photocleavage to enrich for phosphorylated polypeptides.

44. The method of claim 1, further comprising analysing the products of photocleavage to identify phosphorylation sites

45. The method of claim 44, wherein the analysis is a quantitative determination of the degree of phosphorylation at phosphorylated sites.

46. The method of claim 44, wherein the analysis involves mass spectrometry or N-terminal sequencing.

47. The method of claim 1, wherein the uranyl-polypeptide sample is irradiated while immobilized on the IMAC column.

48. The method of claim 47, wherein both the eluate released from the IMAC by photocleavage and the remaining phosphorylated polypeptides on the IMAC column are analysed.

49. A method of purifying a phosphorylated protein from a sample, wherein the phosphorylated protein is immobilized on a column via uranyl coordinated to phosphorylated sites on the protein.

50. The method of claim 49, comprising the steps of:
a. Providing a column with affinity for uranyl;
b. Providing a sample comprising a phosphorylated polypeptide;
e. Providing a sample comprising uranyl;
d. Adding the samples of step b and step c to the column of step a under conditions allowing immobilization of the phosphorylated peptide; and
e. Removing non-binding polypeptides of the sample, and thereby purifying the phosphorylated protein.

51. The method of claim 49, wherein the sample comprising the phosphorylated polypeptide is added to the sample comprising uranyl, after the resulting sample is added to the column with affinity for uranyl.

52. The method of claim 49, wherein the sample comprising uranyl is first added to the column, after the sample comprising phosphorylated polypeptide is added to the column with affinity for uranyl.

53. The method of claim 49, wherein the column is an IMAC column that binds uranyl.

54. The method of claim 49, wherein the column is a phosphate column that binds uranyl.

55. The method of claim 49 further comprising a step of eluting the immobilized protein.

56. The method of claim 49, wherein the sample comprising a phosphorylated polypeptide is a cell extract or is derived from a cell extract.

57. The method of claim 49, wherein the sample comprises a genetically engineered polypeptide with an artificially introduced phosphorylation site for directed cleavage.

58. The method of claim 57, wherein the genetically engineered polypeptide makes up more than 80% w/w of the total polypeptides of the sample.

59. The method of claim 58, wherein the genetically engineered polypeptide further comprises an affinity tag.
